(19) United States
(12) Patent Application Publication
(10) Pub. No.: US 2026/0069634 A1
(43) Pub. Date: Mar. 12, 2026

(54) PHARMACEUTICAL COMPOSITION, FOR PREVENTING OR TREATING HEARING LOSS OR TINNITUS, COMPRISING MITOCHONDRIA AS ACTIVE INGREDIENT

(71) Applicants: PAEAN BIOTECHNOLOGY INC., Daejeon (KR); SEOUL NATIONAL UNIVERSITY R&DB FOUNDATION, Seoul (KR)

(72) Inventors: Young Cheol Kang, Wonju-si, Gangwon-do (KR); Kyuboem Han, Daejeon (KR); Chun-Hyung Kim, Sejong (KR); Soomin Kim, Seoul (KR); Min-Hyun Park, Seoul (KR); Ho Sun Lee, Seoul (KR)

(21) Appl. No.: 18/858,945

(22) PCT Filed: Apr. 27, 2023
(86) PCT No.: PCT/KR2023/005820

§ 371 Date: Oct. 22, 2024

(30) Foreign Application Priority Data
Apr. 27, 2022 (KR) 10-2022-0051833

(51) Int. Cl. A61K 35/28; A61P 27/16

(52) U.S. Cl. CPC A61K 35/28; A61P 27/16

(57) ABSTRACT

Provided is a pharmaceutical composition for preventing or treating hearing loss or tinnitus, the composition comprising mitochondria as an active ingredient. The pharmaceutical composition has the effects of preventing the apoptosis of inner ear cells due to damage and inhibiting hearing loss, and thus can effectively relieve or treat hearing loss or tinnitus.

### TECHNICAL FIELD

The present disclosure relates to a pharmaceutical composition for preventing or treating hearing loss or tinnitus comprising mitochondria as an active ingredient.

### BACKGROUND ART

The auricle and the external auditory meatus are classified as the outer ear, the eardrum and the ossicles auditory are classified as the middle ear, and the cochlea and auditory nerve are classified as the inner ear. Sound is acoustic energy that is transmitted through the auricle and the external auditory meatus to vibrate the eardrum, and the vibration of the eardrum generates mechanical energy that is transmitted to the auditory ossicles. The stapes, the last bone of the auditory ossicles, is connected to the cochlea and transfers the transmitted energy to the lymph fluid within the cochlea. The energy transferred to the lymph fluid causes waves in the lymph fluid, by which hair cells in the cochlea are stimulated. As the movement of hair cells causes ion changes, neurotransmitters are transferred to the auditory nerve attached to the hair cells, and sound energy is transmitted from the auditory nerve to the brain in the form of electrical energy.

When the outer and middle ears, organs that transmit sound, are affected by diseases such as inflammation, they can be recovered through treatment or surgery in most cases, and the resulting hearing impairment can also be improved through treatment. This type of hearing loss is called conductive hearing loss. Meanwhile, sensorineural hearing loss refers to hearing loss caused by problems in the cochlea, an organ that detects sound, the auditory nerve that transmits sound in the form of electrical energy, and the brain that plays a comprehensive role in sound discrimination and understanding. Among the sense organs in the body, the auditory organ is in charge of the most basic sense for communication, which is one of the most important senses for acquiring language and knowledge, and living a human life in society. Most hearing loss belong to sensorineural hearing loss, for which there is no treatment other than prevention to date. Once hearing loss occurs, an assistant means such as a hearing aid is used or a mechanical device is implanted. Additionally, the hearing loss may be accompanied by tinnitus.

Korean Patent Application No. 10 -2008-0044810 discloses that an extract of Scutellaria baicalensis has a significant effect for preventing hearing damage in an animal model of noise-induced hearing loss. In addition, antioxidants, NMDA (N-methyl-D-aspartate) antagonists, apoptosis inhibitors, growth factors, etc. are being studied as effective substances for preventing or treating hearing loss. However, these substances have shown limitations in advancing to clinical trials.

Meanwhile, mitochondria are organelles essential for the survival of eukaryotic cells, which are involved in the synthesis and regulation of adenosine triphosphate (ATP) as an energy source. Mitochondria are important organelles involved in various metabolic pathways in vivo, such as cell signaling, cell differentiation, and cell death, as well as the control of the cell cycle and cell growth. There has been no research reporting that a pharmaceutical composition comprising mitochondria as an active ingredient may be associated with the treatment of hearing loss or tinnitus.

### DISCLOSURE OF THE INVENTION

### Technical Problem

To solve the above problems, the present inventors have developed a method to fundamentally treat hearing loss by developing a composition for treating hearing loss comprising isolated mitochondria as an active ingredient for the treatment of hearing loss or tinnitus.

### Technical Solution

In one aspect, the present disclosure provides a pharmaceutical composition for preventing or treating hearing loss or tinnitus comprising isolated mitochondria as an active ingredient.

### Advantageous Effects

A pharmaceutical composition for preventing or treating hearing loss or tinnitus comprising mitochondria as an active ingredient has the effect of suppressing inner ear cell death caused by damage and consequently suppressing hearing impairment, thereby effectively alleviating or treating hearing loss or tinnitus.

### BRIEF DESCRIPTION OF THE DRAWINGS

FIG. 1 is a graph showing changes in viability of mouse inner ear cells after treatment with an antibiotic (gentamicin and kanamycin), an anticancer agent (cisplatin), or hydrogen peroxide.

FIG. 2 is a graph showing changes in viability of mouse inner ear cells damaged by an antibiotic (gentamicin) after treatment with mitochondria.

FIG. 3 is a graph showing changes in viability of mouse inner ear cells damaged by an antibiotic (kanamycin) after treatment with mitochondria.

FIG. 4 is a graph showing changes in reactive oxygen species generated from mouse inner ear cells damaged by an antibiotic (gentamicin) after treatment with mitochondria.

FIG. 5 is a graph showing changes in reactive oxygen species generated from mouse inner ear cells damaged by an antibiotic (kanamycin) after treatment with mitochondria.

FIG. 6 is a graph showing the degree of hearing recovery in a noise-induced hearing loss model after treatment with mitochondria.

### BEST MODE FOR CARRYING OUT THE INVENTION

Therapeutic agent for treating hearing loss comprising isolated mitochondria as an active ingredient

In one aspect, the present disclosure provides a pharmaceutical composition for preventing or treating hearing loss or tinnitus comprising isolated mitochondria as an active ingredient.

As used herein, the term “mitochondria” is a double membrane-bound organelle found in most eukaryotes that produces a majority of intracellular adenosine triphosphate (ATP).

As used herein, the term “isolated mitochondria” refers to mitochondria obtained from autologous, allogeneic, or xenogeneic sources.

As used herein, the term “autologous mitochondria” refers to mitochondria obtained from plasma, tissue, bone marrow or cells of the same subject. Additionally, the term “allogeneic mitochondria” refers to mitochondria obtained from plasma, tissue, bone marrow, platelets or cells of a subject that belongs to the same species and has a different genotype for alleles. Additionally, the term “xenogeneic mitochondria” refers to mitochondria obtained from plasma, tissue, bone marrow or cells of a subject that belongs to a different species.

The subject may be a mammal, and preferably may be a human.

The mitochondria may be isolated from cells of a subject. The mitochondria may be obtained from autologous or heterologous cells cultured in vitro. The cells may have normal biological activity.

As used herein, the term “cell” refers to a structural or functional unit constituting a living organism, consisting of cytoplasm surrounded by a cell membrane, and containing biomolecules such as proteins and nucleic acids. The cell may refer to a cell containing mitochondria inside the cell membrane.

In addition, the mitochondria may be used by concentrating and disrupting tissues or cells, and then isolating them, or they may be isolated from tissue or cell samples that have been thawed and disrupted after being kept frozen.

The mitochondria may be intact, disrupted, or a combination thereof. In one embodiment, even when the mitochondria are in a disrupted form, they can exhibit pharmacological effects as long as they retain mitochondrial activity.

In one embodiment, the cells may be any one selected from the group consisting of stem cells, somatic cells, germ cells, and platelets.

As used herein, the term “stem cell” refers to an undifferentiated cell that has the ability to differentiate into various types of tissue cells. The stem cells may be any one selected from the group consisting of mesenchymal stem cells, adult stem cells, induced pluripotent stem cells, embryonic stem cells, bone marrow stem cells, neural stem cells, and tissue-derived stem cells.

The mesenchymal stem cells may be derived from any one selected from the group consisting of umbilical cord, umbilical cord blood, bone marrow, fat, muscle, nerve, skin, amniotic membrane, and placenta. Preferably, it may be derived from human umbilical cord.

As used herein, the term “somatic cell” refers to a cell that makes up an organism, excluding a germ cell. The somatic cell may be any one selected from the group consisting of muscle cells, hepatocytes, fibroblasts, epithelial cells, nerve cells, adipocytes, osteocytes, periosteal cells, white blood cells, lymphocytes, and mucosal cells. Preferably, the somatic cells may be obtained from muscle cells or hepatocytes with excellent mitochondrial activity. Additionally, it may be obtained from autologous or heterologous blood PBMC cells.

As used herein, the term “germ cell” refers to a cell that forms a zygote during reproduction in an organism undergoing sexual reproduction. The mitochondria may be obtained from autologous or heterologous germ cells. The germ cells may be sperms or eggs.

As used herein, the term “platelet” refers to a solid component that plays an important role in blood coagulation by bundling fibrin up in the blood to form a blood clot. The mitochondria may be obtained from autologous or heterologous platelets.

Additionally, the isolated mitochondria may have normal biological activity. Specifically, as used herein, mitochondria with normal biological activity may have one or more properties from the group consisting of (i) having a membrane potential, (ii) generating ATP in the mitochondria, and (iii) removing ROS or reducing the activity of ROS in the mitochondria.

In one embodiment, it was confirmed that the pharmaceutical composition of the present disclosure comprising isolated mitochondria as an active ingredient inhibits inner ear cell death when administered to a subject. Therefore, the pharmaceutical composition of the present disclosure effectively alleviates or treats hearing loss or tinnitus.

As used herein, the term “tinnitus” refers to a disease of auditory perception without an actual auditory stimulus. The perceived sound may be a clicking, buzzing, hissing, or roaring sound. The tinnitus is generally associated with hearing loss.

The tinnitus may be caused by hearing loss, otitis media, heart disease, vascular disease, Meniere's disease, brain tumor, acoustic neuroma, migraine, temporomandibular joint disorder, exposure to drugs, head injury, earwax, stress, or depression. Additionally, the tinnitus may be accompanied by hearing loss.

As used herein, the term “hearing loss” refers to a disease characterized by hypoacusis or hearing impairment. The hearing loss may be conductive hearing loss or sensorineural hearing loss.

As used herein, the term “conductive hearing loss” refers to hearing loss that occurs when sound waves are not transmitted normally due to disorders in organs such as the outer ear, eardrum, and middle ear.

As used herein, the term “sensorineural hearing loss” refers to hearing loss caused by abnormalities in the function of the cochlea or abnormalities in the auditory nerve or central nervous system that transmits auditory stimuli to the brain.

The sensorineural hearing loss may be congenital hearing loss, noise-induced hearing loss, sudden sensorineural hearing loss, infection-induced hearing loss, traumatic hearing loss, presbycusis, ototoxic hearing loss, autoimmune hearing loss, Meniere's disease, or hearing loss due to nerve damage.

As used herein, the term “noise-induced hearing loss” refers to hearing loss caused by damage to the cochlea due to external noise.

As used herein, the term “sudden sensorineural hearing loss” refers to about 30 dB or more hearing loss over at least three contiguous audiometric frequencies occurring within about three days.

As used herein, the term “infection-induced hearing loss” refers to hearing loss caused by viral infections such as rubella virus and congenital cytomegalovirus (CMV) infection.

As used herein, the term “traumatic hearing loss” refers to hearing loss resulting from perilymph fistula or pneumolabyrinth caused by trauma.

As used herein, the term “presbycusis” refers to hearing loss caused by degenerative changes due to aging.

As used herein, the term “autoimmune hearing loss” refers to hearing loss caused by autoimmune diseases.

As used herein, the term “Meniere's disease” refers to a disease characterized by simultaneous occurrence of symptoms such as vertigo, hearing impairment, tinnitus, and a feeling of fullness in the ears.

As used herein, the term “hearing loss due to nerve damage” refers to hearing loss caused by abnormalities in any nerve region ranging from the auditory cells of the cochlea to the area responsible for hearing in the brain.

As used herein, the term “ototoxic hearing loss” refers to hearing loss caused by a decline in inner ear function due to drugs, chemicals, or radiation. In one embodiment, the ototoxic hearing loss may be caused by the use of anticancer agents, antibiotics, or radiation. Specifically, the ototoxic hearing loss may be caused by an increase in the concentration of ROS in inner ear cells due to the use of anticancer agents, antibiotics, or radiation, which causes damage to the inner ear.

As used herein, the term “anticancer agent” refers to a drug that inhibits the proliferation of cancer cells and causes necrosis of cancer cells. The anticancer agent may cause neurotoxicity.

In addition, the anticancer agent may be one or more selected from the group consisting of taxane-based anticancer agents, platinum-based anticancer agents, and vinca alkaloid-based anticancer agents. Preferably, the anticancer agent may be a platinum-based anticancer agent.

The taxane-based anticancer agent may be one or more selected from the group consisting of docetaxel, oraxol, paclitaxel, ditaxel, taxol, and taxotere.

The vinca alkaloid-based anticancer agent may be one or more selected from the group consisting of vinblastine, vincristine, vindesine, and vinorelbine.

The platinum-based anticancer agent may be one or more selected from the group consisting of cisplatin, carboplatin, and oxaliplatin.

As used herein, the term “antibiotic” refers to a drug that prevents the growth of or kills other microorganisms. The antibiotics may kill or inhibit the growth of bacteria through one or more mechanisms selected from the group consisting of inhibition of cell wall synthesis, change in cell wall permeability, inhibition of protein synthesis, inhibition of nucleic acid synthesis, and inhibition of folic acid synthesis.

The antibiotics may be one or more selected from the group consisting of beta-lactams, sulfonamides, aminoglycosides, tetracyclines, glycopeptides, ansamycins, penicillins, macrolides, streptogramins, cephalosporins, and quinolones. Preferably, the antibiotics may be aminoglycosides.

In one embodiment, the antibiotics may be one or more selected from the group consisting of streptomycin, neomycin, kanamycin, gentamicin, tobramycin, amikacin, netilmicin, dibekacin, sisomycin, livodomycin, and paromomycin.

In one embodiment, the pharmaceutical composition of the present disclosure comprising isolated mitochondria as an active ingredient may reduce the concentration of ROS increased by the use of anticancer agents, antibiotics, or radiation, prevents damage to the inner ear, thereby preventing or treating ototoxic hearing loss or tinnitus.

As used herein, the term “treatment” may be used to encompass both therapeutic treatment and preventive treatment. The prevention can be used to mean alleviating or reducing the pathological condition or disease of a subject.

As used herein, the term “active ingredient” refers to an ingredient that exhibits an activity alone or in combination with an auxiliary agent (carrier) that does not have an activity in itself. In one embodiment, the isolated mitochondria as the active ingredient can inhibit inner ear cell death caused by damage, an increase in ROS, or hearing impairment.

The isolation of mitochondria from a specific cell may be performed by using various known methods, such as using a specific buffer solution or using a potential difference or magnetic field. Additionally, the isolation of mitochondria may comprise centrifuging and filtering plasma to remove all cellular components, and centrifuging the filtered plasma.

The isolation of mitochondria can be performed by disrupting and centrifuging tissues or cells in terms of maintaining the mitochondrial activity. In one embodiment, it may be performed by culturing cells, conducting a first centrifugation of a composition containing the cells to produce pellets, resuspending the pellets in a buffer solution and homogenizing the same, conducting a second centrifugation of the homogenized solution to produce a supernatant, and conducting a third centrifugation of the supernatant to purify the mitochondria. It is preferable in terms of maintaining cell activity that the time for performing the second centrifugation is adjusted to be shorter than the time for performing the first and the third centrifugation. The speed can be increased from the first centrifugation toward the second centrifugation and the third centrifugation.

Specifically, the first to the third centrifugations may be performed at a temperature of about 0° C. to about 10° C., preferably at a temperature of about 3° C. to about 5° C. In addition, the time for which the centrifugation is performed may be about 1 to 50 minutes, and may be appropriately adjusted depending on the number of centrifugations and the content of the sample.

In addition, the first centrifugation may be performed at a speed of about 100×g to about 1,000×g, about 200×g to about 700×g, or about 300×g to about 450×g. Additionally, the secondary centrifugation may be performed at a speed of about 1×g to about 2,000×g, about 25×g to about 1,800×g, or about 500×g to about 1,600×g. Additionally, the third centrifugation may be performed at a speed of about 100×g to about 20,000 ×g, about 500×g to about 18,000×g, or about 800×g to about 15,000×g.

In addition, pharmaceutically acceptable saccharides may be used to stabilize the obtained mitochondria. Specifically, it may be sucrose, mannitol, trehalose, etc., but is not limited thereto. In addition, a pharmaceutically acceptable tris, HEPES, phosphate, etc. without limitation may be used as a buffer.

Meanwhile, after obtaining the mitochondria, additives such as chelators and antioxidants can be used to remove damage caused by ion leakage and suppress oxidative stress. The additives may include any reagent widely known in the art without limitation. For example, it may be EDTA, EGTA, citrate, glycine, taurine, ATP, etc., but is not limited thereto.

In addition, the isolation of mitochondria may be performed by thawing, disrupting, and centrifuging frozen cells or tissues in terms of maintaining the mitochondrial activity. The method for obtaining mitochondria may be performed by freezing cells or tissues, thawing the cells or tissues, and disrupting the thawed cells and tissues.

The freezing temperature is not limited thereto, but may be −1° C. or lower. Specifically, the temperature may be about −5° C. to about −200° C., about −15° C. to about −180° C., about −25° C. to about −160° C., about −40° C. to about −140° C., about −55° C. to about −120° C., about −60° C. to about −100° C., or about −70° C. to about −90° C.

The freezing may be performed using liquid nitrogen (LN2) or a refrigerator. Specifically, the refrigeration or freezing process can be performed by rapid freezing using LN2 or by using a refrigerator such as a deep freezer, a freezer, or a freezing container. The freezing is not particularly limited as long as it can refrigerate or freeze mitochondria.

The freezing time is not particularly limited as long as the frozen mitochondria have normal activity.

The isolated mitochondria can be quantified by quantifying the membrane proteins of the mitochondria. Specifically, the isolated mitochondria can be quantified using BCA (bicinchoninic acid) assay. The mitochondria may be included in the pharmaceutical composition at a concentration of 0.1 μg/ml to 1,000 μg/ml, 1 μg/ml to 750 μg/ml, 25 μg/ml to 500 μg/ml, 25 μg/ml to 150 μg/ml, or 25 μg/ml to 100 μg/ml. In one embodiment, the mitochondria are used at a concentration of 25 μg/ml or 50 μg/ml.

Additionally, the number of the isolated mitochondria can be measured using a particle counter (Multisizer 4e, Beckman Coulter).

In one embodiment, the mitochondria may be included in the pharmaceutical composition in an amount of 1×10⁵ mitochondria/ml to 9×10⁹ mitochondria/ml. Specifically, the mitochondria may be included in the pharmaceutical composition in an amount of 1×10⁵/ ml to 5×10⁹/ml, 2×10⁵/ml to 2×10⁹/ml, 5×10⁵/ml to 1×10⁹/ml, 1×10⁶/ml to 5×10⁸/ml, 2×10⁶/ml to 2×10⁸/ml, 5×10⁶/ml to 1×10⁸/ml or 1×10⁷/ml to 5×10⁷/ml.

The pharmaceutical composition may further comprise a pharmaceutically acceptable carrier. The pharmaceutically acceptable carrier may be any carrier that is a non-toxic material suitable for delivery to a patient. Distilled water, alcohol, fats, waxes, or inert solids may be included as a carrier. Pharmacologically acceptable adjuvants (buffers, dispersants) may also be included in the pharmaceutical composition.

Specifically, the pharmaceutical composition may be prepared into a parenteral formulation depending on the route of administration by a conventional method known in the art, including a pharmaceutically acceptable carrier in addition to an active ingredient. “Pharmaceutically acceptable” means that not having more toxicity than a subject to be applied (prescribed) can tolerate without inhibiting the activity of the active ingredient.

When the pharmaceutical composition of the present disclosure is prepared as a parenteral formulation, it can be formulated in the form of injections, transdermal drugs, and nasal inhalers according to methods known in the art using a suitable carrier. When formulated as an injection, suitable carriers may include sterilized water, ethanol, polyols such as glycerol or propylene glycol, or mixtures thereof. Preferably, Ringer's solution, phosphate buffered saline (PBS) containing triethanolamine, or sterile injectable water, or isotonic solutions such as 5% dextrose can be used.

The pharmaceutical composition of the present disclosure may be an injectable preparation. Therefore, in order to ensure product stability for the distribution of injections, the pharmaceutical composition of the present disclosure may be prepared as an injection that is very stable physically and chemically by adjusting the pH using a buffer solution such as an aqueous acid solution or phosphate that can be used for an injection.

Specifically, the pharmaceutical composition of the present disclosure may comprise water for injection.

The water for injection is distilled water made to dissolve solid injections or reconstitute water-soluble injections, which may be glucose injection, xylitol injection, D-mannitol injection, fructose injection, saline solution, Dextran 40 injection, Dextran 70 injection, amino acid injection, Ringer's solution, lactic acid-Ringer's solution, or a phosphate buffer solution or sodium dihydrogen phosphate-citric acid buffer solution with a pH ranging from about 3.5 to about 7.5.

The pharmaceutical composition of the present disclosure may further comprise a stabilizer or a solubilizing agent. For example, the stabilizer may be pyrosulfite or ethylene diaminetetraacetic acid, and the solubilizing agent may be hydrochloric acid, acetic acid, potassium hydroxide phosphate, potassium bicarbonate, potassium carbonate, or tris. In one embodiment, the pharmaceutical composition may comprise a mixed preservative solution such as TTG (trehalose-tris-glycine) solution, which can be commonly used in pharmaceutically acceptable drug preparations.

Specifically, the pharmaceutical composition of the present disclosure may comprise a liquid composition for injection in addition to isolated mitochondria. The isolated mitochondria are as described above. The pharmaceutical composition of the present disclosure comprising the liquid composition for injection can be used as a composition for preventing or treating diseases associated with mitochondrial function. In addition, administration of mitochondria through injection can suppress the formation of blood clots that may be caused by mitochondrial aggregation, reduction and aggregation of platelets, etc., maintain and/or strengthen mitochondrial stability, and stably maintain mitochondrial activity.

The liquid composition may comprise glycine, saccharides, and buffer.

The glycine may be present in the liquid composition for injection at a concentration of about 15 mM or more, specifically at a concentration of about 15 mM to about 150 mM, about 17 mM to about 130 mM, about 20 mM to about 120 mM, about 22 mM to about 110 mM, or about 25 mM to about 100 mM, but is not limited thereto. In addition, the glycine can be used with one or more amino acids which is selected from the group consisting of histidine, isoleucine, leucine, lysine acetate, methionine, phenylalanine, threonine, tryptophan, valine, alanine, arginine, asparaginic acid, cysteine, glutamic acid, proline, serine, and tyrosine, but is not limited thereto.

In addition, the saccharide included in the liquid composition for injection is not limited thereto, but may be one or more selected from the group consisting of sucrose, trehalose, mannitol, sorbitol, glucose, fructose, mannose, maltose, lactose, isomaltose, dextran, and dextrin. In particular, the saccharide may be trehalose, mannitol or sucrose. Preferably, the saccharide may be trehalose.

The buffer included in the liquid composition for injection is not limited thereto, but may be selected from the group consisting of tris buffer, HEPES buffer, MOPS buffer, and acetate-containing or phosphate-containing buffer. Preferably, the buffer may be an injectable tris buffer.

The pH of the buffer is not limited thereto, but may range from about 7.0 to about 7.8, from about 7.2 to about 7.6, or from about 7.3 to about 7.5.

In addition, the buffer is not limited thereto, but may be present in the liquid composition for injection at a concentration of about 5 mM to about 50 mM, about 8 mM to about 40 mM, about 10 mM to about 35 mM, about 13 mM to about 30 mM or about 15 mM to about 25 mM.

The liquid composition for injection may have an osmolarity of about 200 to about 400 mOsm, about 230 to about 380 mOsm, about 250 to about 350 mOsm, about 260 to about 320 mOsm, about 270 to about 330 mOsm, or about 280 to about 300 mOsm. The osmolarity in the above range promotes long-term storage at a temperature of 2° C. to 8° C. or higher, and renders the composition suitable for parenteral administration, such as intravascular, intramuscular, or subcutaneous administration, without causing side effects in a subject.

As used herein, the term “osmolarity” refers to the moles of solute contributing to the osmotic pressure of the solution per kilogram of solvent. Osmolarity is determined by measuring the freezing point depression of a sample using an osmometer.

Additionally, the liquid composition for injection may further comprise a chelating agent.

The chelating agent is not limited thereto, but may be one or more selected from the group consisting of injectable grades of EGTA, EDTA, and BAPTA. The chelating agent can remove damage due to ion leakage after obtaining mitochondria for the liquid composition for injection.

Additionally, the pharmaceutical composition may comprise additives such as antioxidants, ATP, magnesium, etc., which are effective in maintaining the function and activity of mitochondria.

The pharmaceutical composition of the present disclosure may be stored in a container selected from the group consisting of vials, cartridges, syringes and autoinjectors. Additionally, the container in which the pharmaceutical composition is stored may be stored at room temperature, at a refrigeration temperature of about 2° C. to about 8° C., or at a temperature of about 25° C. to about 40° C. until administered to a subject in need of treatment.

The subject is not limited thereto, but may be a mammal such as a human, dog, cow, horse, pig, sheep, goat, cat, mouse, rabbit, or rat, and preferably may be a human.

The pharmaceutical composition of the present disclosure is administered in a pharmaceutically effective amount. The term “therapeutically effective amount” or “pharmaceutically effective amount” refers to an amount of a compound or composition effective in preventing or treating hearing loss, which is sufficient to treat the disease at a reasonable benefit/risk ratio applicable to medical treatment and does not cause side effects. The level of the effective amount may be determined by factors including the patient's health status, type and severity of the disease, drug activity, sensitivity to the drug, administration method, administration time, administration route and excretion rate, treatment period, combined or concurrent use of drugs, and other factors well known in the medical field. In one embodiment, a therapeutically effective amount refers to an amount of drug that is effective in treating or preventing hearing loss.

As used herein, the term “administration” means introducing a predetermined substance into a subject by an appropriate method, and the composition may be administered through any general route as long as it can reach the target tissue. The pharmaceutical composition may be administered intratympanically, intraperitoneally, intravenously, intramuscularly, subcutaneously, intradermally, topically, intranasally, or rectally, but is not limited thereto. The intratympanic administration may be a direct injection into the tympanic chamber or administration through surgery (e.g., tympanostomy).

The preferred dosage of the pharmaceutical composition of the present disclosure per one administration may be about 0.01 mg mitochondria/kg to about 5 mg mitochondria/kg, about 0.1 mg mitochondria/kg to about 4 mg mitochondria/kg, or about 0.25 mg mitochondria/kg to about 2.5 mg mitochondria/kg based on the body weight of the subject to be administered, but is not limited thereto. It is most preferable in terms of cell activity that the pharmaceutical composition be administered such that isolated mitochondria are administered in an amount within the above range based on the body weight of a subject suffering from hearing loss. Additionally, the pharmaceutical composition can be administered 1 to 10 times, 3 to 8 times, or 5 to 6 times, and preferably 5 times. The administration interval can be 1 to 7 days or 2 to 5 days, and preferably 3 days. Such a dosage should not be construed as limiting the scope of the invention in any aspects.

The term “subject” refers to an individual to which the composition of the present disclosure can be applied (prescribed), and may be a subject suffering from hearing loss. Additionally, the subject may be a mammal such as a human, rat, mouse, or livestock, but preferably a human.

In one embodiment, the pharmaceutical composition may further comprise a known preventive or therapeutic agent for hearing loss, and administration of the pharmaceutical composition may be combined with additional hearing loss treatment.

Another aspect of the present disclosure provides a method of treating and/or preventing hearing loss comprising administering isolated mitochondria to a subject.

In addition, the pharmaceutical composition may further comprise a known agent for preventing or treating hearing loss, and administration of the pharmaceutical composition may be combined with additional hearing loss treatment.

### Mode for Carrying Out the Invention

Hereinafter, the present disclosure will be described in more detail by the following examples. However, the following examples are only for illustrating the present disclosure, and the scope of the present disclosure is not limited to these only.

### Preparation Example 1

### Preparation of Mitochondria

Umbilical cord-derived mesenchymal stem cells stored at a concentration of 1×10⁷ cells/ml were physically disrupted using pressure and homogenized, and then subjected to the first centrifugation at a speed of 1,100×g. Mitochondria were obtained using a differential centrifugation method that isolates mitochondria from the cells by centrifuging the supernatant at a speed of 12,000×g for 15 minutes at 4° C.

### Example 1

### Determination of Viability of Mouse Inner Ear Cells Upon Treatment With Antibiotics or Anticancer Agents

### Example 1.1

### Culture of Mouse Inner Ear Cells

Mouse-derived inner ear cells (House Ear Institute-Organ of Corti 1, HEI-OC1) were cultured in a DMEM (Dulbecco's Modified Eagle Medium, Gibco) medium containing 10% fetal bovine serum (FBS, Gibco), 100 μg/ml streptomycin, and 100 U/ml ampicillin.

### Example 1.2

### Determination of Viability of Inner Ear Cells

The cell viability was determined after the mouse inner ear cells obtained in Example 1.1 were treated with an antibiotic or an anticancer agent. Specifically, 6×10³ mouse inner ear cells (House Ear Institute-Organ of Corti 1) (HEI-OC1) were cultured in a DMEM medium, and 16 hours later, the cells were treated with gentamicin (2 mM, 4 mM, 6 mM, 8 mM and 10 mM), cisplatin (5 mM, 10 mM, 15 mM, 20 mM, 25 mM and 50 mM), kanamycin (5 mM, 10 mM, 15 mM and 20 mM) or H₂O₂ (25 mM, 50 mM or 100 mM) for 24 hours for each concentration. The cell viability was measured using the WST-1 assay.

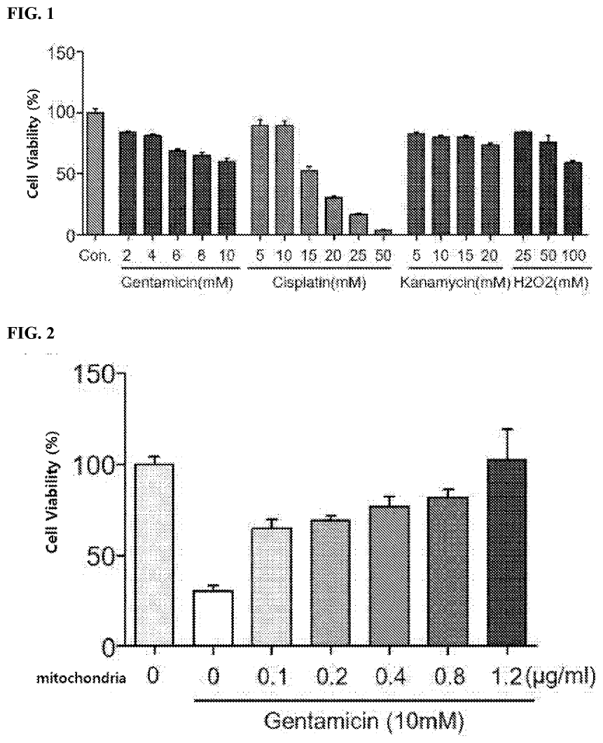

As shown in FIG. 1, the viability of inner ear cells decreased in a concentration-dependent manner in all groups treated with gentamicin, cisplatin, kanamycin, or H₂O₂ as compared to the group not treated with a drug (Con.). The results mean that hearing loss may be caused by damage to inner ear cells by a drug or reactive oxygen species.

### Example 2

### Confirmation of Reduction in Damage Caused by Antibiotics Upon Treatment With Mitochondria

### Example 2.1

### Confirmation of Recovery From Decrease in Cell Viability Caused by Antibiotics

It was investigated whether mitochondrial treatment had an effect in recovering the decrease in viability in the inner ear cells damaged by drug treatment in Example 1.2.

Specifically, 6×10³ mitochondrial mouse inner ear cells (House Ear Institute-Organ of Corti 1) (HEI-OC1) were cultured in an antibiotic DMEM medium. 16 hours later, the cells were treated with 10 mM gentamicin or 16.2 mM kanamycin for 24 hours, and then treated with the mitochondria of Preparation Example 1 at a concentration of 0 μg/ml, 0.1 μg/ml, 0.2 μg/ml, 0.4 μg/ml, 0.8 μg/ml, and 1.2 μg/ml for 24 hours. The viability of inner ear cells after treatment was measured using the WST-1 assay.

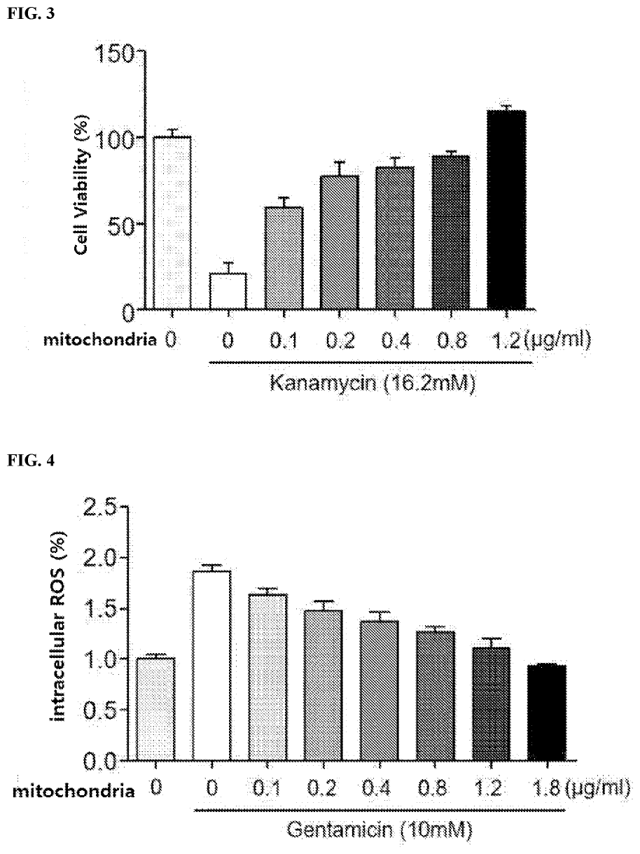

As shown in FIGS. 2 and 3, it was confirmed that the cell viability was recovered when treated with mitochondria as compared to the group not treated with mitochondria (0 μg/ml). In addition, this recovery effect was achieved in a concentration-dependent manner, depending on the mitochondrial concentration.

### Example 2.2

### Confirmation of Effect of Suppressing Increase in Reactive Oxygen Species Caused by Antibiotics

It was investigated whether mitochondrial treatment had an effect in suppressing the increase in reactive oxygen species in the inner ear cells damaged by drug treatment in Example 1.2.

Specifically, 6×10³ inner ear cells (House Ear Institute-Organ of Corti 1) (HEI-OC1) were cultured in a DMEM medium, and 16 hours later, the cells were treated with 10 mM gentamicin or 20 mM kanamycin. 24 hours after the treatment, the cells were treated with mitochondria. The group treated with gentamicin was treated with the mitochondria of Preparation Example 1 at a concentration of 0 μg/ml, 0.1 μg/ml, 0.2 μg/ml, 0.4 μg/ml, 0.8 μg/ml, 1.2 μg/ml, and 1.8 μg/ml, respectively. The group treated with kanamycin was treated with the mitochondria of Preparation Example 1 at a concentration of 0 μg/ml and 0.4 μg/ml, respectively. 24 hours later, the treated medium was stained with DCF-DA (Invitrogen), a staining reagent specific for intracellular reactive oxygen species, for 1 hour, and then the reactive oxygen species were measured using a Plate Reader (Biotek, synergy HTX). In addition, the number of the cells was corrected after staining with Hoechst33432 (Invitrogen), a staining reagent specific for nucleus.

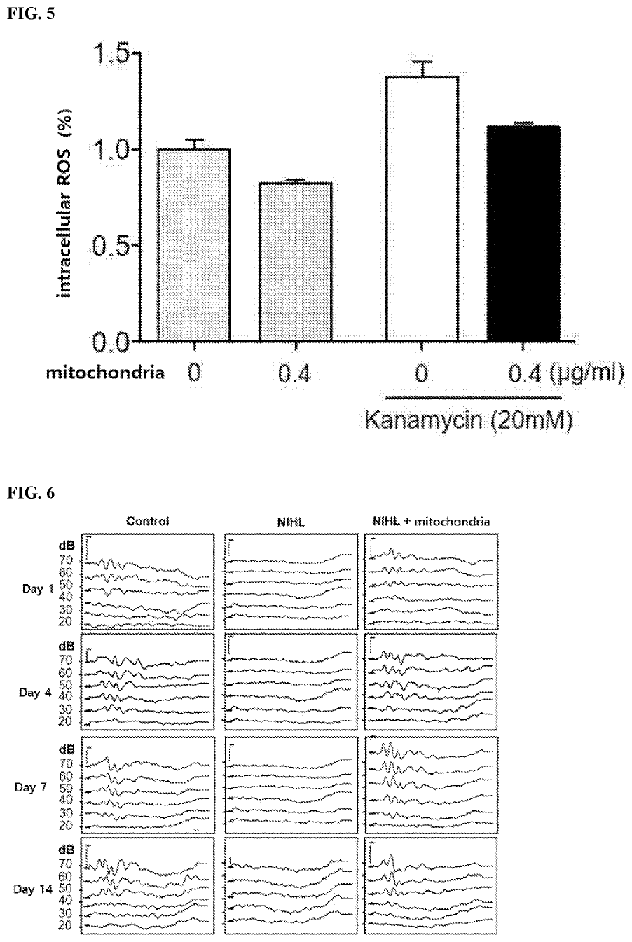

As shown in FIGS. 4 and 5, it was confirmed that the rate of increase in reactive oxygen species was low in the group treated with mitochondria as compared to the group not treated with mitochondria (0 μg/ml). In addition, it was confirmed that intracellular reactive oxygen species increased by drug treatment were reduced to the normal level by mitochondrial treatment.

### Example 3

Confirmation of Hearing Recovery Effect by mitochondrial Treatment in Noise-Induced Hearing Loss Animal Model

An experimental group in which hearing loss was induced by noise and a control group in which hearing loss was not induced were prepared using 12-week-old C57BL6 female mice. Specifically, 12-week-old C57BL6 female mice in the experimental group were exposed to 120 dB sound for 2 hours. In addition, 20 μg of mitochondria obtained from Preparation Example 1 were injected into the caudal vein twice before and after the exposure to 120 dB sound for 2 hours. Hearing loss was not induced in the control group, and it was not treated with mitochondria. Auditory brainstem responses (ABR) were measured in the control and experimental groups 1, 4, 7, and 14 days after injection.

As shown in FIG. 6, mice with noise-induced hearing loss had no response to sounds of 20 to 70 dB, but the experimental group administered with mitochondria showed responses to sounds of 30 to 70 dB, like the control group. These results mean that mitochondria have the effect of restoring hearing from noise-induced hearing loss.

1. A pharmaceutical composition for preventing or treating hearing loss or tinnitus comprising isolated mitochondria as an active ingredient.

2. The pharmaceutical composition for preventing or treating hearing loss or tinnitus according to claim 1, wherein the mitochondria are isolated from cells.

3. The pharmaceutical composition for preventing or treating hearing loss or tinnitus according to claim 2, wherein the cells are somatic cells, germ cells, stem cells, blood cells, or a combination thereof.

4. The pharmaceutical composition for preventing or treating hearing loss or tinnitus according to claim 3, wherein the stem cells are mesenchymal stem cells, induced pluripotent stem cells, embryonic stem cells, or a combination thereof.

5. The pharmaceutical composition for preventing or treating hearing loss or tinnitus according to claim 4, wherein the mesenchymal stem cells are derived from umbilical cord, umbilical cord blood, bone marrow, fat, muscle, nerve, skin, amniotic membrane, placenta, synovial fluid, testis, periosteum, or a combination thereof.

6. The pharmaceutical composition for preventing or treating hearing loss or tinnitus according to claim 1, wherein the isolated mitochondria have normal activity.

7. The pharmaceutical composition for preventing or treating hearing loss or tinnitus according to claim 1, wherein the hearing loss is conductive hearing loss or sensorineural hearing loss.

8. The pharmaceutical composition for preventing or treating hearing loss or tinnitus according to claim 7, wherein the sensorineural hearing loss is noise-induced hearing loss, sudden sensorineural hearing loss, infection-induced hearing loss, traumatic hearing loss, presbycusis, ototoxic hearing loss, autoimmune hearing loss, Meniere's disease, or hearing loss due to nerve damage.

9. The pharmaceutical composition for preventing or treating hearing loss or tinnitus according to claim 8, wherein the ototoxic hearing loss is caused by the use of anticancer agents, antibiotics, or radiation.

10. The pharmaceutical composition for preventing or treating hearing loss or tinnitus according to claim 9, wherein the anticancer agent is one or more selected from the group consisting of taxane-based anticancer agents, platinum-based anticancer agents, and vinca alkaloid-based anticancer agents.

11. The pharmaceutical composition for preventing or treating hearing loss or tinnitus according to claim 10, wherein the anticancer agent is a platinum-based anticancer agent.

12. The pharmaceutical composition for preventing or treating hearing loss or tinnitus according to claim 11, wherein the platinum-based anticancer agent is one or more selected from the group consisting of cisplatin, carboplatin, and oxaliplatin.

13. The pharmaceutical composition for preventing or treating hearing loss or tinnitus according to claim 9, wherein the antibiotic is one or more selected from the group consisting of streptomycin, neomycin, kanamycin, gentamicin, tobramycin, amikacin, netilmicin, dibekacin, sisomycin, livodomycin, and paromomycin.

14. The pharmaceutical composition for preventing or treating hearing loss or tinnitus according to claim 1, wherein the isolated mitochondria inhibit inner ear cell death.

15. The pharmaceutical composition for preventing or treating hearing loss or tinnitus according to claim 1, wherein the mitochondria are contained at a concentration of 0.1 μg/ml to 1,000 μg/ml in the pharmaceutical composition.

16. A method of preventing or treating hearing loss or tinnitus comprising administering the pharmaceutical composition according to claim 1 to a subject.

17. Use of the pharmaceutical composition according to claim 1 for preventing or treating hearing loss or tinnitus.
